(19) United States
(12) Patent Application Publication
(10) Pub. No.: US 2006/0034934 A1
(43) Pub. Date: Feb. 16, 2006

(54) Agglomeration of sterol particles

(76) Inventors: Matthew L. DeGuise, Wayzata, MN (US); Luke A. Stevens, Minneapolis, MN (US)

Correspondence Address: CARGILL, INCORPORATED, LAW/24, 15407 MCGINTY ROAD WEST, WAYZATA, MN, 55391, US

(21) Appl. No.: 10/918,766

(22) Filed: Aug. 13, 2004

(51) Int. Cl. A61K 9/14; A61K 31/56

(52) U.S. Cl. 424/489; 514/169

(57) ABSTRACT

Disclosed are agglomerates of sterol particles comprising sterol particles having a mean particle size of equal to or less than about 100 microns. Also disclosed are methods for producing the agglomerates of sterol particles that include contacting sterol particles with a liquid, or a liquid and binder, and drying the resultant dispersion.

### FIELD OF THE INVENTION

This invention relates to processes for the production of agglomerates of sterol particles having improved flow properties, and to the resultant agglomerates of sterol particles.

### BACKGROUND

There are presently available sterol particles having a mean particle size of approximately 10 microns. This fine particle size is necessary for performance in certain food and beverage applications to overcome the hydrophobic nature and high melting point of the sterol particles. Moreover, sterol particles in a fine sized form may be more available in the gut for cholesterol blocking.

It is also known, however, that the fine particle size sterol particles have poor flow properties and exhibit a tendency to form a cake upon storage, and in bulk handling. This is disadvantageous in certain dietary supplement applications such as tablet pressing and hard-shell capsule filling where flow within the filling machines is important for proper machine operation and a consistent fill weight.

Therefore, it is apparent that it is difficult to have sterol particles with a fine particle size that are also free flowing.

It would be desirable therefore to have fine powdered sterol particles that are free flowing, and processes for producing the sterol particles.

### SUMMARY OF THE DISCLOSURE

The present disclosure relates in one embodiment, to an agglomerate of sterol particles having a mean particle size of equal to or less than about 100 microns. The agglomerate preferably has a mean particle size of about 150 microns to about 850 microns. Also, the present disclosure relates, in various embodiments, to methods for producing the agglomerates that comprise contacting the sterol particles, optionally with agitation, with a liquid in which the sterol particles are not more than about 1% soluble and that is removable from the sterol particles by drying, or with an organic binder that is at least about 1% soluble in a liquid that is removable from sterol particles by drying and with a liquid in which the sterol particles are not more than about 1% soluble and that is removable from the sterol particles by drying, until a dispersion is produced, and removing the liquid from the dispersion by drying.

### DETAILED DESCRIPTION OF THE DISCLOSURE

The present disclosure relates in one embodiment, to an agglomerate of sterol particles having a mean particle size of equal to or less than about 100 microns. The agglomerate preferably has a mean particle size of about 150 microns to about 850 microns. Also, the present disclosure relates, in various embodiments, to methods for producing the agglomerates that comprise contacting the sterol particles, optionally with agitation, with a liquid in which the sterol particles are not more than about 1% soluble and that is removable from the sterol particles by drying, or with an organic binder that is at least about 1% soluble in a liquid that is removable from sterol particles by drying and with a liquid in which the sterol particles are not more than about 1% soluble and that is removable from the sterol particles by drying, until a dispersion is produced, and removing the liquid from the dispersion by drying.

The novel agglomerates of the present disclosure, in some embodiments, are comprised of sterol particles having a mean particle size of equal to or less than about 100 microns, preferably less than about 70 microns, more preferably less than about 40 microns, and still more preferably, less than about 20 microns. The agglomerates, in some embodiments, preferably have a mean particle size of about 150 microns to about 850 microns. The term sterol particles as used herein means both specific sterol particles such as sitosterol, campesterol, stigmasterol, brassicasterol, avenasterols, and diosgenin, or mixtures of specific sterol particles. The specific sterol particles or mixtures of sterol particles or sterol particles derivatives may be isolated from the following sources: oilseeds such as soybeans, canola seed, corn, sunflower, cottonseed, palm kernel, corn fiber, soy germ, sheanut, or peanut; tree sources such as tall oil, tall oil soap or tall oil pitch; other plant sources such as Mexican yam, olives, or sugar cane. Also included within the definition of sterol particles are hydrogenated forms of the above mentioned sterol particles (known in the art as stanols) including, but not limited to, sitostanol and campestanol. Further included within this definition are ester derivatives of sterol particles such as steryl or stanol fatty acid esters, ferulate esters, or succinate esters. Also included within this definition are sterol particle based pharmaceuticals and pharmaceutical intermediates such as estron, estrogen, progesterone, testosterol, androstenedione, androstene-diene-dione. Mixtures of all of the various sterol particles are also within the scope of the disclosure.

In producing the agglomerates of the sterol particles herein, two methods are described as follows:

In a first embodiment, sterol particles having a mean particle size of equal to or less than about 100 microns, are optionally subjected to agitation. Any known manner for agitation may be utilized such as, for example, a laboratory scale food processor, or a commercial scale blender or high shear granulator. The sterol particles are contacted with a liquid that is removable from the sterol particles by drying. Any liquid that is removable from the sterol particles and in which the sterol particles are not more than 1% soluble, is suitable for use in the method. Exemplary of liquids suitable for use, but not limited thereto, are water, ethanol, mixtures of water and ethanol, water/ethyl acetate mixture, and mixtures thereof, and the like. Preferred for use as the liquid herein is water. The liquid is utilized herein in an amount ranging from about 1 part liquid to about 99 parts sterol particles, to about 99 parts liquid to about 1 part sterol particles. As used herein, all reference to parts is intended to define parts by weight. A preferred amount ranges from about 1 part liquid to about 4 parts sterol particles, to about 4 parts liquid to about 1 part sterol particles, with a more preferred amount ranging from about 1 part liquid to about 2 parts sterol particles, to about 2 parts liquid to about 1 part sterol particles. The contact with the liquid, and the optional agitation, are continued until a resulting dispersion of sterol particles and liquid is formed. Thereafter, the dispersion is dried, in the presence or absence of further agitation, at any temperature lower than that at which the sterol particles melt, to remove the liquid from the dispersion. The product remaining, after the removal of the liquid, is the agglomerate of sterol particles.

In another embodiment of the disclosure, the method for producing the agglomerate of sterol particles comprises contacting the sterol particles with an organic binder that is at least about 1% soluble in a liquid that is removable from sterol particles by drying, and, with a liquid in which the sterol particles are not more than about 1% soluble and that is removable from the sterol particles by drying. More particularly, in this embodiment, sterol particles having a mean particle size of equal to or less than about 100 microns, are optionally subjected to agitation. The sterol particles are contacted with an organic binder that is at least about 1% soluble in the liquid that is removable from the sterol particles by drying, and a liquid in which the sterol particles are not more than about 1% soluble and that is removable from the sterol particles by drying. Examples of suitable organic binders include, but are not limited to, any food grade component, such as maltodextrin, a natural and/or artificial sugar, a sugar polyol, a vitamin, for example Vitamin C (ascorbic acid), Vitamin D, or the like, a binder polymer, for example, hydroxypropyl cellulose, polyvinylpyrrolidone, and mixtures thereof, or the like. Any liquid that can be removed from the sterol particles by drying, and in which not more than 1% of the sterol particles are soluble, and in which the binder is at least about 1% soluble, may be used herein. Examples of suitable liquids include, but are not limited to, water, ethanol, mixtures of water and ethanol, ethyl acetate/water mixture, and mixtures thereof, and the like. A preferred liquid is water. In this method, where both an organic binder, and a liquid that can be removed by drying are used, the organic binder is present in an amount ranging from about 1 part binder to about 1000 parts sterol particles, to about 100 parts binder to about 1 part sterol particles; and the liquid is present in an amount ranging from about 1 part liquid to about 3 parts sterol particles, to about 99 parts liquid to about 1 part sterol particles. As mentioned previously herein, all reference to parts is intended to define parts by weight. Preferably there is used about 1 part liquid to about 2 parts sterol particles, to about 4 parts liquid to about 1 part sterol particles, and more preferably about 1 part liquid to about 2 parts sterol particles, to about 1 part liquid to about 1 part sterol particles. In respect of the binder component, in one embodiment, there may be utilized an amount ranging from about 1 part binder to about 100 parts sterol particles, to about 1 part binder to about 1 part sterol particles, and in another embodiment, an amount of about 1 part binder to about 50 parts sterol particles, to about 1 part binder to about 10 parts sterol particles. In this process, the sterol particles are contacted, optionally while being agitated, with the organic binder, and the liquid that can be removed from the sterol particles by drying. The liquid is optionally heated to about the boiling point, at which point the binder is added to the liquid to dissolve and form a solution. The solution is added to the sterol particles and the optional agitation is continued until there is produced a dispersion. Thereafter, the dispersion is dried, for example in a vacuum oven, in the presence or absence of further agitation, to remove the liquid from the dispersion. The product remaining after the removal of the liquid, is the agglomerate of sterol particles.

The agglomerates of sterol particles produced by the methods herein may be roughly ground, if necessary, and/or classified, if desired. During the production of the agglomerates of the sterol particles, the agglomerates, in several embodiments, may be classified to obtain agglomerates of sterol particles that preferably have a particle size of about 150 microns to about 850 microns. The classification may be carried out using any conventional method and apparatus for the classification of particles, such as, for example, using screen classification technique having screens of varying sizes. The agglomerates of sterol particles having a particle size smaller than about 150 microns may preferably be recycled for further agglomeration. The agglomerates of sterol particles having a particle size greater than about 850 microns may preferably be recycled for further grinding. The grinding may be achieved by any conventional technique, such as by using a hammer mill, a cone mill, a jet mill, or the like.

As the data in the Examples show, the agglomerates of the sterol particles of the present invention are characterized by having increased poured bulk density, and enhanced free flowing characteristics and, correspondingly, are less subject to pluggage. The agglomerates of sterol particles herein having improved flow properties are expected to be advantageous in applications where flow properties are important, such as in the manufacture of pressed tablets and in capsule filling, in the dietary supplement market. Further, it is expected that the agglomerates of sterol particles herein, having improved flow properties, will improve the handling characteristics associated with incorporation of sterol particles into food and beverages.

In the methods of the present disclosure, if desired, other conventional additives may be incorporated in the preparation of the agglomerates of sterol particles. Exemplary of suitable additives are those that are soluble in the liquid used in the process. Included are soluble nutrition ingredients, for example vitamins, minerals, herbals and the like; soluble coloring agents; soluble flavoring agents; soluble surfactants, for example polysorbates, lecithin, monoglycerides and the like, and mixtures thereof.

The methods for producing the agglomerates sterol particles in the examples herein comprise subjecting a sterol particle to agitation using a Black and Decker Power Pro 2 (FP1500) food processor. There may be used any other suitable means such as a mixer, a ribbon blender, a high shear agglomerator, or the like. Thereafter, a liquid in which the sterol particle is not more than about 1% soluble and that can be removed from the sterol particle by drying, in this instance water, heated to near the boiling point, is slowly added to the agitated sterol particles. The agitation, and addition of liquid, continue until there is produced a dispersion. Where an organic binder is used, the binder is added to the agitated sterol particles, with the liquid described above, the water, in which the binder is at least about 1% soluble, and the process is followed until a dispersion is produced. In those instances where both a binder, and a liquid that can be removed from sterol particles by drying, are utilized, the binder and the liquid can be added to the agitated sterol in any known manner. For example, the binder and liquid can be added separately to the agitated sterol particles; or the binder can be dry mixed with the agitated sterol particles and then the liquid is introduced to the dry mix of binder and sterol particles; or the binder can be premixed with the liquid to form a solution or dispersion, that is then added to the agitated sterol particles; and the like. In the examples herein, wherein both a binder and a liquid were incorporated, the binder was dissolved in the water, that had been heated to about the boiling point, to produce a solution that was added to the agitated sterol particles. The resulting sterol particles, contacted with liquid or binder-liquid dispersion, are then dried to yield dried agglomerates of sterol particles. In this instance, the dried agglomerates of sterol particles were reground in the food processor.

In preparing the dried agglomerates of sterol particles herein, any suitable method for achieving the drying may be utilized. The following examples utilized two types of drying techniques. In Examples 3-7 herein, the sterol particles that have been contacted with either a liquid, or a dispersion comprising an organic binder and a liquid, were dried in a vacuum oven at a temperature of about 80° C. and under a vacuum of about 50 mm (millimeters) of mercury (2 inches mercury). Alternatively, in Examples 8-12, the sterol particles that have been contacted with a dispersion comprising an organic binder and a liquid, were dried by placing the samples, in the 5 L glass tub of a Sherwood Scientific Laboratory Model Fluid Bed Dryer, at a temperature of about 82° C., for a period of about 45 minutes, and a blower set to a velocity of 3.10 m/sec (meters/second).

The invention will be more readily understood by reference to the following examples. There are, of course, many other forms of this invention which will become obvious to one skilled in the art, once the invention has been fully disclosed, and it will accordingly be recognized that these examples are given for the purpose of illustration only, and are not to be construed as limiting the scope of this invention in any way.

### EXAMPLES

In the following examples, the properties of the agglomerates of sterol particles were determined in accordance with the following test procedures.

POURED BULK DENSITY—The poured bulk density is determined by placing a funnel in a tared 250 ml graduated cylinder. The product to be measured is poured into the cylinder through the funnel, and the funnel is removed. The net mass of product added is calculated. The volume of product added is estimated. From this data, the poured bulk density is calculated.

MEAN PARTICLE SIZE—The mean particle size is determined using a Laser Scattering Particle Size Distribution Analyzer Model LA-910, available from Horiba Company. Prior to analysis, dried sterol particles of each of Examples 1-12 herein was agitated with water and Triton X-100 surfactant, that is available from E. M. Science (CAS 9002-93-1). The resultant product was then sonicated for a period of about 5 minutes using an Ultrasonic Cleaner Model FS30H, available from Fisher Scientific, wherein the water bath temperature was maintained at a temperature of about 45° C. The resultant sonicated product was then treated in a Tenbroeck Tissue Grinder, Model KT885000-0007, available from Kontes Glass Company. The tissue grinder was plunged three (3) times when utilized. The resultant dispersion was then placed into the Model LA-910 Particle Size Distribution Analyzer, and the manufacturer's instructions were followed in determining the mean particle size of the samples. The Analyzer was operated by sonicating, prior to analysis, for a period of about 15 minutes, then analyzing at an agitation level of 2, and at a circulation level of 6.

FLOW—General flow characteristics are qualitatively indicated by visual observation.

### Examples 1-12

The samples of Examples 3-12 were prepared using the procedures herein described. There was utilized a particular drying technique for Examples 3-7, and another drying technique for Examples 8-12. The drying techniques are described herein. The conditions used in preparing each of Examples 3-12 are reported in the following Table I. Further, the evaluation of the properties of each of Examples 1-12 were determined in accordance with the Test Methods described herein, and the results of the evaluations are also reported in Table I.

In Examples 1-12, the sterol particles utilized were CoroWise FP-100, trademarked sterol particles produced and sold by Cargill, Incorporated. The sterol particles comprise sitosterol (40-58%), campesterol (20-28%), stigmasterol (14-23%), brassicasterol, campestanol, beta-sitostanol, delta-5-avenasterol, and other sterol particles. In Examples 1, and 3-7, the starting sterol particles were CoroWise FP-100 sterol having a mean particle size of 11.8 microns. In Examples 2, and 8-12, the starting sterol particles was CoroWise FP-100 sterol particles having a mean particle size of 10.6 microns.

TABLE I

| | Water | Binder | | | | | | | |
|---|---|---|---|---|---|---|---|---|---|
| | Usage | Usage | | Sterol | | | Mean | Poured | |
| | (g water/ | (g binder/ | | Particles | Water | Binder | Particle | Bulk | Flow |
| Example | g sterol | g sterol | | Amount | Amount | Amount | Size | Density | (Visual |
| No. | particles) | particles) | Binder Type | (g) | (g) | (g) | (μ) | (g/ml) | Observation) |
| 1 (control) | 0 | 0 | None | — | 0 | 0 | 11.8 | 0.24 | Poor |
| 2 (control) | 0 | 0 | None | — | 0 | 0 | 10.6 | — | Poor |
| 3 | 0.27 | 0.05 | Maltodextrin¹ | 195.4 | 53.1 | 10.8 | 15.6 | 0.23 | Slightly |
| | | | | | | | | | improved over |
| | | | | | | | | | control |
| 4 | 0.19 | 0.04 | Maltodextrin¹ | 196.1 | 37.2 | 7.8 | 15.3 | 0.22 | Slightly |
| | | | | | | | | | improved over |
| | | | | | | | | | control |
| 5 | 0.8 | 0 | None | 195.2 | 157.1 | 0 | 14.5 | 0.34 | Much |
| | | | | | | | | | improved |
| | | | | | | | | | over control |
| 6 | 0.76 | 0.05 | Maltodextrin¹ | 196.4 | 149.8 | 10.3 | 13.9 | 0.38 | Nearly free |
| | | | | | | | | | flowing |
| 7 | 0.77 | 0.01 | Maltodextrin¹ | 197.6 | 163.7 | 2.18 | 12.9 | 0.32 | Nearly free |
| | | | | | | | | | flowing |
| 8 | 0.80 | 0.05 | Maltodextrin¹ | 50 | 40 | 2.5 | 19.6 | 0.28 | Nearly free |
| | | | | | | | | | flowing |
| 9 | 0.76 | 0.04 | Gum Arabic² | 50.2 | 38 | 2 | 14.9 | 0.37 | Free flowing |
| 10 | 0.80 | 0.04 | Polyvinyl- | 50 | 40 | 2 | 15.1 | 0.38 | Free flowing |
| | | | pyrrolidone | | | | | | |
| 11 | 0.79 | 0.04 | TWEEN 80 | 50.4 | 40 | 2 | 12.2 | 0.47 | Nearly free |
| | | | Surfactant³ | | | | | | flowing |
| 12 | 0.64 | 0.04 | Arabinogalactan | 50.5 | 32.5 | 1.9 | 16.2 | 0.36 | Free flowing |

¹Maltodextrin utilized is available from Grain Processing Corporation under the trademark MALTRIN M250
²Gum Arabic utilized is available from TIC Gums
³TWEEN 80 surfactant is available from BASF, and is a polysorbate having a molecular weight of 80,000

From the above data, it is apparent that the agglomerates of sterol particles produced by several processes of the present disclosure (Examples 5-12) were characterized both by an increase in poured bulk density, and a significant improvement in discernable flowability, as compared to the sterol particles of Examples 1 and 2, that were not agglomerated. Examples 3 and 4 of Table 1 are agglomerates of sterol particles produced by a process where the amount of water and/or binder were outside the present disclosure. The data show that the agglomerates of Example 3 and 4 were characterized by slightly lower poured bulk densities and only slight improvement in flow properties, as compared to the sterol particles of Examples 1 and 2 that were not agglomerated.

A further observation from the data in Table 1, is related to the mean particle size of the agglomerates of sterol particles. It is significant that when the agglomerates of sterol particles are redispersed in water, the mean particle size of the resulting sterol particles was, in general, similar to the mean particle size of the starting sterol particles that was utilized in producing the agglomerates of sterol particles.

Therefore, it is apparent that the present disclosure relates to agglomerates that, when utilized in various applications, results in the presence of sterol particles having both a fine particle size and good flow properties.

The foregoing has been a description of an illustrative embodiment of the present invention. The present invention is not to be limited in scope by the illustrative embodiments described which are intended as specific illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims.

What is claimed is:

1. An agglomerate of sterol particles comprising sterol particles having a mean particle size of equal to or less than about 100 microns.

2. The agglomerate of sterol particles in accordance with claim 1 wherein the mean particle size of the sterol particles is less than about 70 microns.

3. The agglomerate of sterol particles in accordance with claim 1 wherein the mean particle size of the sterol particles is less than about 20 microns.

4. The agglomerate of sterol particles in accordance with claim 1, said agglomerate having a mean particle size of about 150 microns to about 850 microns.

5. A method for producing agglomerates of sterol particles comprising:
(a) contacting sterol particles having a mean particle size of equal to or less than about 100 microns with a liquid that is removable from the sterol particles by drying and in which the sterol particles are not more than 1% soluble, until a dispersion is produced; and
(b) drying the dispersion to remove liquid from the dispersion, whereby the agglomerates of sterol particles are obtained.

6. The method in accordance with claim 5 further comprising agitating the sterol particles.

7. The method in accordance with claim 5 wherein the liquid is selected from the group consisting of water, ethanol, a mixture of water and ethanol, a mixture of water and ethyl lactate, and mixtures thereof.

8. The method in accordance with claim 7 wherein the liquid is water.

9. The method in accordance with claim 5 further comprising agitating the dispersion during drying.

10. The method in accordance with claim 5 further comprising incorporating an additive.

11. The method in accordance with claim 6 further comprising grinding the agglomerates of sterol particles.

12. The method in accordance with claim 5 further comprising classifying the agglomerates.

13. The method in accordance with claim 5 wherein the liquid is present in an amount ranging from about 1 part liquid to about 99 parts sterol particles, to about 99 parts liquid to about 1 part sterol particles, wherein parts are parts by weight.

14. The method in accordance with claim 5 wherein the liquid is present in an amount ranging from about 1 part liquid to about 4 parts sterol particles, to about 4 parts liquid to about 1 part sterol particles, wherein parts are parts by weight.

15. A method for producing agglomerates of sterol particles comprising:
(a) contacting the sterol particles having a mean particle size of equal to or less than about 100 microns with a liquid that is removable from the sterol particles by drying and in which the sterol particles are not more than 1% soluble, and an organic binder that is at least 1% soluble in the liquid, until a dispersion is produced; and
(b) drying the dispersion to remove liquid from the dispersion, whereby the agglomerates of sterol particles are obtained.

16. The method in accordance with claim 15 further comprising agitating the sterol particles.

17. The method in accordance with claim 15 wherein the liquid is selected from the group consisting of water, ethanol, a mixture of water and ethanol, a mixture of water and ethyl acetate, and mixtures thereof.

18. The method in accordance with claim 15 wherein the liquid is water.

19. The method in accordance with claim 15 wherein the organic binder is selected from the group consisting of maltodextrin, a natural sugar, an artificial sugar, a sugar polyol, a mineral, a vitamin, a binder polymer, gum arabic, polyvinylpyrrolidone, a polysorbate surfactant, arabinogalactan, and mixtures thereof.

20. The method in accordance with claim 15 wherein the organic binder is selected from the group consisting of maltodextrin and gum arabic.

21. The method in accordance with claim 15 further comprising incorporating an additive.

22. The method in accordance with claim 15 further comprising grinding the agglomerates of sterol particles.

23. The method in accordance with claim 15 further comprising classifying the agglomerates.

24. The method in accordance with claim 15 wherein the organic binder is present in an amount ranging from about 1 part binder to about 1000 parts sterol particles, to about 100 parts binder to about 1 part sterol particles, wherein parts are parts by weight.

25. The method in accordance with claim 15 wherein the organic binder is present in an amount ranging from about 1 part binder to about 100 parts sterol particles, to about 1 part binder to about 1 part sterol particles, wherein parts are parts by weight.

26. The method in accordance with claim 15 wherein the liquid is present in an amount ranging from about 1 part liquid to about 3 parts sterol particles, to about 99 parts liquid to about 1 part sterol particles, wherein parts are parts by weight.

27. The method in accordance with claim 15 wherein the organic binder is dry mixed with the sterol particles prior to contacting the sterol particles with the liquid.

28. The method in accordance with claim 15 wherein the organic binder is premixed with the liquid, prior to contacting the sterol particles with the binder.

29. The method in accordance with claim 15 further comprising incorporating an additive selected from the group consisting of a soluble nutrition ingredient, a vitamin, a mineral, a herbal, a coloring agent, a flavoring agent, a surfactant, a polysorbate, a lecithin, a monoglyceride, and mixtures thereof.

30. An agglomerate of sterol particles produced in accordance with the method of claim 5.

31. An agglomerate of sterol particles produced in accordance with the method of claim 15.

32. The agglomerate of sterol particles in accordance with claim 30, having a mean particle size of about 150 microns to about 850 microns.

33. The agglomerate of sterol particles in accordance with claim 31, having a mean particle size of about 100 mesh to about 20 mesh.
